(19) United States
(12) Patent Application Publication
(10) Pub. No.: US 2012/0330203 A1
(43) Pub. Date: Dec. 27, 2012

(54) Orthopedic Knee Brace Supplemental Support

(76) Inventors: Timothy Jones, Austin, TX (US); Jennifer Clark, Austin, TX (US)

(21) Appl. No.: 13/312,602

(22) Filed: Dec. 6, 2011

Related U.S. Application Data
(60) Provisional application No. 61/499,444, filed on Jun. 21, 2011.

(51) Int. Cl. A61F 5/00

(52) U.S. Cl. 602/16

(57) ABSTRACT

Disclosed is a supplemental support that is designed to prevent a knee brace from migrating out of position. The device comprises an inelastic, elongated strap with hook fasteners on its inward surface and loop fasteners on its outward surface adapted to connect a knee brace to a waist support. A loop is provided at the upper end of the strap for securing to a conventional waist belt, while a protective cover secures over any exposed length of strap to prevent chaffing or damage to clothing. The hook fasteners secure to the neoprene fabric or any exposed loop fasteners on a conventional knee brace, or alternatively the strap is inserted between the brace straps and the neoprene to ensure that the brace does not move along the user's leg. When positioned between a waist belt and a knee brace, the device ensures the brace remains in position during activity.

### CROSS REFERENCE TO RELATED APPLICATION

This application claims the benefit of U.S. Provisional Application No. 61/499,444 filed on Jun. 21, 2011, entitled “Orthopedic Knee Brace Suspender.”

### BACKGROUND OF THE INVENTION

1. Field of the Invention

The present invention relates to orthopedic braces. More specifically, the present invention pertains to a support or suspender for an orthopedic knee brace to prevent the brace from changing position or creeping down a user's leg while the user is in motion.

Trauma to the knee is a very common orthopedic injury. Examples of knee injuries include bone fractures and breaks, ligament stretches and tears, twisting beyond the knee's allowable range of motion, injuries to the muscles and tendons surrounding the joint, and inflammation of the bursa. Knee injuries typically require rest, surgery, rehabilitation, or a combination thereof. Such procedures generally require the use of a stabilizing device to secure the knee joint and limit or prevent movement, such as a compression sleeves and hinged knee support braces.

A majority of conventional knee braces presently available do not include a means of preventing the device from migrating out of position along the leg of the patient, particularly when the user is mobile. A brace that moves out of position allows for excess movement of the knee, and does not provide ample support thereto. This can lead to further injury, potentially prolonging rehabilitation or introducing a risk of additional surgery.

The present invention is designed to supplement a conventional knee brace by supporting the brace from a waist support while not interfering with its original operation or requiring any modification thereto. The present invention comprises an inelastic, elongated strap designed to bridge the knee brace and a waist support, connecting each and preventing the brace from sliding downwards, particularly for active users. Situated along the strap length are hook fasteners on its inward surface and loop fasteners on its outward surface, a loop at its upper end for securing to a waist belt and a protective cover that secures over the exposed strap length to prevent abrasion of the belt, the user's clothes or skin. The hook fasteners secure to the neoprene fabric or to any exposed loop fasteners on the knee brace, while the belt connection at the strap upper end is provided in a loop form or hook for accepting the belt therein or therethrough. Additionally, straps provided on the brace may further be secured over the connection between the present invention and the brace to ensure that the connection remains intact during operation. This provides a secure connection between the user and the brace, thereby ensuring that the brace remains in position and does not slide down the user's leg as the knee is extended and flexed.

2. Description of the Prior Art

Several devices have been disclosed in the art that attempt to secure a knee brace in position on a user's leg to prevent migration out of position. Lamping, U.S. Pat. No. 6,485,448 discloses a knee strap comprised of an elongated flexible member having a tubular member secured to one surface thereof in a cloth pouch, a buckle on one end of the strap which cooperates with the other end of the strap to secure the strap around a human leg, the tubular member being positioned to enable the tubular member to bear immediately below the patella while the buckle bears on the side of the knee, and not the front or back. This device is secured into position below a user's knee for additional support by way of a nonslip tubular member, and does not include any additional attachment means to secure the brace into position.

Other devices in the art attempt to hold a knee brace in position on a user with an additional fastening means. Nathanson, U.S. Pat. No. 7,967,765 discloses an apparatus and method of securing an orthopedic brace on a wearer such that the brace is unlikely to migrate. In preferred embodiments, the apparatus includes a band that extends entirely around the wearer's leg, making contact with the entire circumference thereof. In certain embodiments, a strap secured to the brace threads through loops on the band, thereby operably connecting the brace and the band. In certain other embodiments, the apparatus further includes a belt that overlaps the band. Suspenders operably connect the brace to the band/belt. Because the band makes contact with the entire circumference of the wearer, the apparatus comfortably secures the brace to the wearer while effectively preventing migration of the brace and without hindering the intended function of the brace. While this device is designed to prevent a brace from moving out of position, its means of attachment is considerably different from the present invention. The additional securing point is in the upper quadriceps region, and does not attach to a user's waist belt to prevent migrating as does the present invention.

Other devices disclosed in the art attempt to secure a knee brace in position by utilizing a means of attaching to a belt. Billet, U.S. Pat. No. 5,785,673 discloses a brace for supporting a weakened knee joint wherein lower and upper anchor bands are carried by a sleeve for location upon and attachment to the lower leg and the upper leg adjacent the knee joint, with essentially inextensible support straps following respective helical paths around the knee joint between the lower and upper anchor bands, and inflatable elements juxtaposed with the lower and upper anchor bands which subsequently are inflated to anchor the attached lower and upper anchor bands in place for firm and accurate securement upon installation in the desired location and for maintaining the desired location during use. While this device includes an attachment means that utilizes a waist belt, it requires the use of the waist belt attached to the device and does not allow a user to affix the knee brace to his or her own belt. In addition, the waist belt attachment utilizes clips for attachment thereto, which do not allow the same level of adjustability as does the present invention.

The devices disclosed in the prior art have several known drawbacks. These devices attempt to prevent migration by utilizing nonslip materials, additional attachment means, or by attachment to a specially designed waist band, and further require the use of a proprietary knee brace. The present invention overcomes these limitations by attaching to any conventional knee brace, and requiring no modification thereto to connect to the support. The present device bridges the gap between a waist support and a knee brace by providing a strap having fastening that attaches to the neoprene or exposed loop fasteners on the brace, and connects over the waist support at its upper end. The support from the waist and the inelasticity of the strap prevents the brace from migrating out of position. The design of the present invention substantially diverges in design elements from the prior art and consequently it is clear that there is a need in the art for an improvement to existing orthopedic knee devices. In this regard the instant invention substantially fulfills these needs.

### SUMMARY OF THE INVENTION

In view of the foregoing disadvantages inherent in the known types of orthopedic knee devices now present in the prior art, the present invention provides a new supplemental orthopedic knee device wherein the same can be utilized for providing convenience for the user when stabilizing and preventing movement in a knee for the purpose of rehabilitation.

It is therefore an object of the present invention to provide a new and improved supplemental orthopedic knee device that has all of the advantages of the prior art and none of the disadvantages.

Another object of the present invention is to provide a new and improved supplemental orthopedic knee brace support device that connects from a waist support to an existing knee brace to prevent the brace from migrating out of position while in use, wherein the brace will not migrate down the user's leg while the user is actively articulating the knee joint.

Another object of the present invention to provide a new and improved supplemental orthopedic knee brace support device that attaches to a conventional knee brace having either a neoprene material or material affixable to hook fasteners. Straps provided on the brace may provide further securement of the knee brace support.

Yet another object of the present invention is to provide a new and improved supplemental orthopedic knee brace support device that forms a belt attachment means at its upper end that secures to a waist belt or strap, supporting the knee brace support while positioned between the waist and knee brace.

Still another object of the present invention is to provide a new and improved supplemental orthopedic knee brace support device that includes a protective covering to prevent snagging on clothing, such as knits and silk, and prevention of chaffing of the support device against the leg of a user.

Yet another object of the present invention is to provide a new and improved supplemental orthopedic knee brace support device that can further include a waist belt.

A final object of the present invention is to provide a new and improved supplemental orthopedic knee brace support device that is inexpensive and simple to manufacture.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the present invention, including an elongated strap, a closed loop and a protective covering.

FIG. 2 shows a perspective view of the present invention installed on a conventional knee brace and connected to a waist support.

FIG. 3 shows a perspective view of the present invention in use in conjunction with a conventional knee brace and a waist support while the user is in motion.

### DETAILED DESCRIPTION OF THE INVENTION

Reference is made herein to the attached drawings. Like reference numerals are used throughout the drawings to depict like or similar elements of the supplemental knee brace support. For the purposes of presenting a brief and clear description of the present invention, the preferred embodiment will be discussed as used for attaching to a conventional knee brace to prevent migration out of position. The figures are intended for representative purposes only and should not be considered to be limiting in any respect.

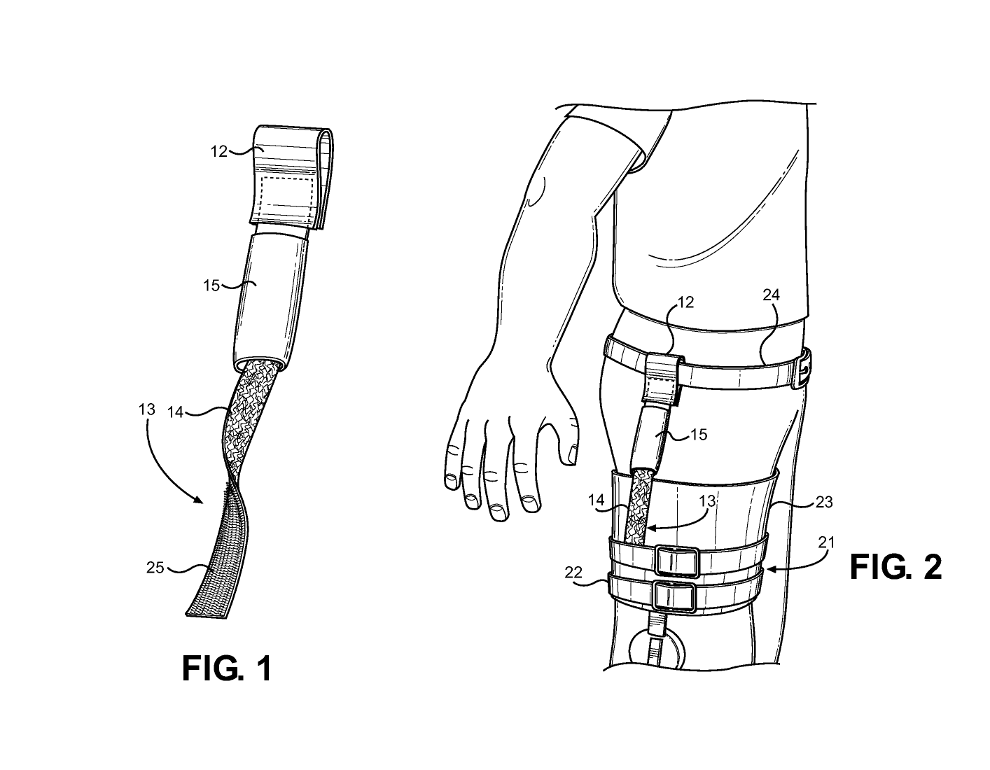

Referring now to FIG. 1, there is shown a perspective view of the present invention, comprising an elongated strap 13 with a securing means on its inward 25 and outward 14 faces and a waist support loop 12 at its upper terminus. The device is intended to attach to any conventional knee brace and provide vertical securement that prevents migration of the knee brace out of position. The waist support loop 12 comprises an attached loop, hook or closed section of the strap folded onto itself and secured together. The loop 12 functions to connect the strap to a belt or over the top of a user's pant waist, supporting the strap 13 at its upper end while attached to a knee brace at its lower end. The working lower end of the device attaches to a knee brace down the leg from a user's belt, spanning the gap between the waist and the knee region. Downward migration of the brace during activity is countered as the strap absorbs load and transfers any motion of the brace to the waist support belt or pant waist to prevent movement.

The elongated strap 11 is preferably constructed of an inelastic material so as to prevent elongation thereof while supporting the knee brace in position. Those of skill in the art will appreciate that the elongated strap 11 may be made from a variety of materials, such as nylon, cotton, leather or alternative synthetic or natural material. The inelasticity prevents the knee brace from migrating out of position by resisting elongation while in connection thereto, as load imparted on the strap transfers directly to the user's belt or pant waist and is prevented from being absorbed through strain energy of the strap. Migration occurs when a user moves about, thereby causing the brace to slide towards the distal portion of the leg. The securing means 25 of the present invention provides connection to a conventional orthopedic knee brace in the way of hook fasteners 14 about the strap inward surface, wherein the hooks provide connectors for fibrous material or loop fastener material on the brace itself. The hook fasteners 25 are provided on the inward surface of the strap 11, and cover its entire length to allow use with different length users and different distances between the user's waist and knee brace support. The outward surface 14 of the strap preferably comprises loop fastener material, which can be used by existing straps on the knee brace as a connection point to the strap.

At the upper terminus of the strap, the waist support loop 12 may comprise several different embodiments. The preferred embodiment of the loop 12 comprises the strap folding onto itself, covered with an exterior material and sewn together. The loop fasteners of the outward surface are placed on the interior of the loop 12 to prevent snagging, while the hook fasteners of the inner surface are covered by the exterior material. This construction is the least material intensive and easiest to manufacture. Alternate embodiments of the loop comprise a material forming a loop at the end of the strap and sewn thereto. Still another embodiment contemplates the use of an open hook, as opposed to a closed loop, wherein the hook provides a rigid connector that attaches to the end of the strap and affords greater flexibility with regards to the size and style of belt utilized in conjunction therewith. Secure construction of the waist support loop 12 is necessary to ensure the present invention will securely support the load applied thereto by the orthopedic knee brace as it moves during flexure and extension of the knee joint. In an alternate embodiment, the present invention can additionally include an attached waist belt that affixes through the loop 12 to prevent the knee brace from migrating out of position. This allows the device to be utilized without a user-provided belt, or a strong pant waist region that would otherwise be utilized to support the device upper end.

To prevent snagging of articles of clothing and chaffing of the user's legs while in use, the length of strap not engaged with the knee brace and not forming the waist support loop 12 is concealed by a protective covering 15. The covering 15 is a closed loop of material that the straps is fed through, which prevents abrading a user's leg or damage to clothing along.

Referring now to FIG. 2, there is shown a perspective view of the present invention installed onto a user and in conjunction with a conventional orthopedic knee brace 21. Conventional knee braces 21 are often made from a synthetic rubber material, such as neoprene, or provide ample locations for attachment of any necessary securing means (in the way of straps of hook and loop fastening or similar attachment means). Additionally, knee braces 21 are often covered with a plurality of hook and loop fasteners for attachment of their own straps around the circumference of the user's leg, with the loop side of the fastener being exposed around the leg circumference and the securing straps of the device having hook fastener regions. This design gives a user a plurality of locations for attachment of the prevent invention elongated strap 13. In one embodiment, the hook fastener on the elongated strap 13 can secure to the neoprene fabric 23 of the knee brace 21 to prevent migration. Alternately, the strap 13 can be inserted between the knee brace straps 22 and the neoprene fabric 23 and secured together to further ensure the strap 13 remains in place. The outer loop fasteners 14 further provide a means for connection of any knee brace straps thereto.

The loop 12 secures to a waist support means, such as belt 24 or alternatively over the top of a user's pant waist region, preventing the brace 21 from sliding towards the distal portion of the leg when installed thereon. A conventional belt 24 supports the device in position and utilizes the user's hips to prevent movement. As can be appreciated by those of skill in the art, a properly secured waist belt cannot migrate towards the distal portion of a user's legs due to the circumferential difference between a user's waist and hipline. The connection made by the loop 12 to a belt 24 anchors the elongated strap 13 on the user. The hook fasteners on the strap 13 secure to the brace 21, thereby creating a secure system that prevents migration of the brace 21. The inelastic properties of the strap 13 material prevent it from stretching under load, thereby further prohibiting brace 21 movement. The protective covering 15 is then installed over the strap, covering its inward and outward surfaces to prevent chaffing and snagging on clothing.

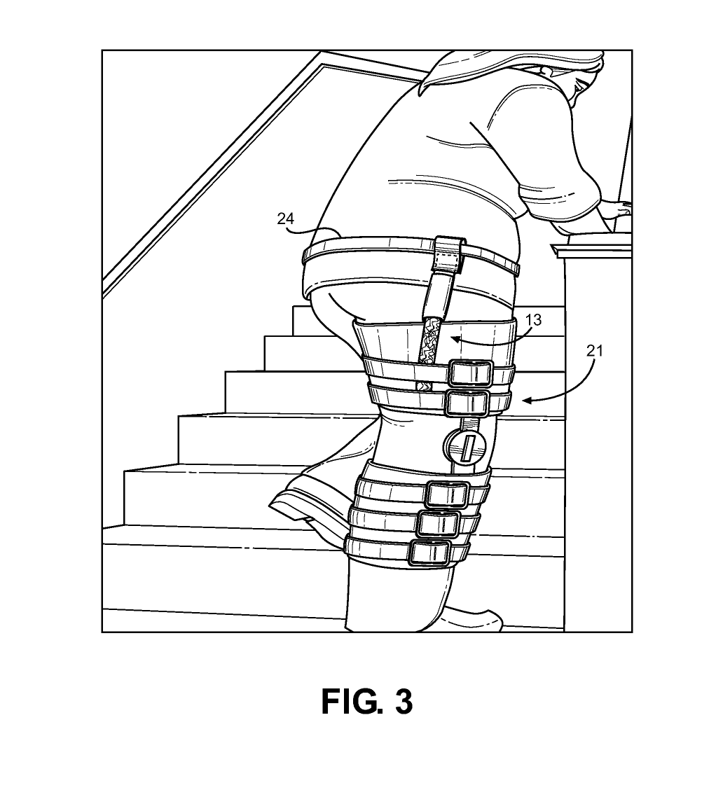

Referring now to FIG. 3, there is shown a perspective view of the present invention installed on a knee brace 21 and in use as the user is moving. As a user moves with the orthopedic knee brace 21 installed, the flexing and extending of the leg pulls the upper portion of the brace 21 downwards toward the distal end of a user's leg. The upper quadriceps region of a leg has a greater circumference than the lower quadriceps region, which similarly has a greater circumference than the gastrocnemius region. By securing the brace 21 to a user's waist belt 24, the present invention overcomes the problem of the brace 21 migrating down the user's leg. During movement, migration and downward movement is resisted, thereby preventing the user from maneuvering with the brace out of position or providing inadequate knee support as prescribed by the brace.

Overall, the present invention provides a user with a safe and convenient device for preventing a knee brace from migrating out of position, and one that can be utilized with any commonly available knee brace available on the market. Its design is universal and can be updated to function with a number of different brace styles without requiring modification of the brace to connect the device thereto. The present invention overcomes knee brace location drifting by properly securing a knee brace to a support location along the user's waist. The hook fasteners along the strap can be secured to a variety of conventional knee braces, and provides connection to a waist support by way of a waist support loop structure. The connection provides an inelastic bridge between the waist and the brace, ensuring that the brace remains in position while walking or otherwise bending the knee. The present invention saves a user time and considerable effort required to unfasten the knee brace in order to reposition it properly around the leg if movement occurs.

Knee supports are required to support a user's knee for a number of different situations. It is contemplated that the present invention provides utility for all common types of knee braces currently in use. Of particular concern are those braces that provide a level of mobility for a user after an operation. The user's mobility places a great deal stabilizing load on the brace in order to allow the knee to be supported while the user walks. It may be otherwise impossible for a user to walk without the brace due to muscle atrophy, or because of any reconstruction that may have occurred in order to fix an injury during an operation. A brace that is utilized for such a purpose can potentially cause further injury to the leg if it migrates towards a user's foot and transfers the stresses intended for the brace back onto the knee of the user. By securing the brace into position, the present invention overcomes such difficulties encountered with conventional knee braces, and affords a user a secure means of preventing brace migration.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An orthopedic knee brace support device, comprising:
an elongated, inelastic strap having inward and outward surfaces, an upper region and a lower region;
said inward surface comprising a hook fasteners along its length;
said upper region having a waist support attachment means.

2. The device as in claim 1, wherein said outward surface comprises loop fasteners along its length.

3. The device as in claim 1, further comprising a protective covering forming a closed loop around said strap and extending along its length.

4. The device as in claim 1, wherein said waist support attachment means comprises a closed loop, creating an aperture to accept a belt therethrough.

5. The device of claim 4, wherein said closed loop is constructed by folding said strap onto itself and connected together, wherein said hook fasteners line said loop interior; said folded strap covered by a section of protective material.

6. The device of claim 4, wherein said closed loop is constructed connecting a closed loop to said strap upper terminus.

7. The device as in claim 1, wherein said waist support attachment means comprises an open hook adapted to attach over a belt or paint waistline.

8. The device as in claim 1, further comprising a waist belt for attachment around a user's waist and wherein said loop comprises a closed loop, creating an aperture to accept said waist belt therethrough.
